(19) United States
(12) Patent Application Publication
(10) Pub. No.: US 2007/0009621 A1
(43) Pub. Date: Jan. 11, 2007

(54) Correcting systemic androgen levels using Eurycoma longifolia

(76) Inventor: Annie Eng, Bradenton, FL (US)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 11/176,464

(22) Filed: Jul. 7, 2005

(51) Int. Cl. A61K 36/18

(52) U.S. Cl. 424/773

(57) ABSTRACT

A method for treating Systemic Androgen Deficiency, by administering Eurycoma longifolia, a plant native to South East Asia which regulates testosterone biosynthesis in vivo by, inter alia, impacting the activity of CYP17 (17-hydroxylase/17,20 lysase) and leutenizing hormone.

### RELATED APPLICATIONS

This application incorporates by reference the teachings of Provisional Application Ser. No. 60/697,099, filed 7 Jul. 2005.

### BRIEF DESCRIPTION

As men age, their level of androgen hormones decreases. This decrease is associated with a number of adverse conditions, including a decrease in the ratio of “good” cholesterol to “bad” cholesterol, and a decrease in the level of insulin growth factor-1. I have identified a class of symptoms which appear to be caused by a systemic deficiency in the amount of available androgen; I denote this class of symptoms, “Systemic Androgen Deficiency.”

I have found that one can avoid or treat Systemic Androgen Deficiency by administering an effective amount of a plant, Eurycoma longifolia.

### BACKGROUND

The art teaches that men over perhaps middle age experience what is often called “andropause,” a term used to describe a male version of menopause, wherein the level of male sex hormones decreases with a person's age.

The art also suggests that erectile dysfunction is a condition perhaps caused by a decreased level of male sex hormones.

The art teaches that androgens are synthesized in vivo via a multi-step pathway, and that androgen levels may decrease if this synthetic pathway is impeded. Androgen levels may decrease, for example, by inadequate CYP17 (17-hydroxylase/17, 20 lyase) or leutenizing hormone activity. It may also be caused by inadequate conversion of androgen precursor compound into its product (e.g., conversion of cholesterol into pregnenolone, or pregnenolone into progesterone, dehydroepiandrosterone, cortisol, or testosterone.

The art also teaches that Sex Hormone Binding Globulin binds testosterone, reducing the amount of free systemic testosterone.

The art teaches that pheromone An-alpha is a human male pheromone, the systemic level of which decreases with age.

Androgen, or male sex hormone, is defined as a substance capable of developing and maintaining masculine sexual characteristics (including the genital tract, secondary sexual characteristics and fertility) and the anabolic status of somatic tissues.

Testosterone is a hormone that has a unique effect on a man's total body. Testosterone is produced in the testes and in the adrenal glands. It is to males what estrogen is to females. Testosterone helps to build protein and is essential for normal sexual behavior and producing erections. It also affects many metabolic activities such as production of blood cells in the bone marrow, bone formation, lipid metabolism, carbohydrate metabolism, liver function and prostate gland growth. Testosterone stimulates metabolism, which promote fat burning, increases the formation of red blood cell, and accelerates muscle growth. When testosterone levels are low, the body tries to compensate by making more cholesterol, a precursor to adrenal testosterone production. Testosterone is synthesized by an enzymatic sequence of steps from cholesterol to Pregnenolone and DHEA via CYP-17 (17-hydroxylase/17,20 lyase), and ultimately converted to testosterone. CYP-17 is involves in the early stage of steroid biosynthesis.

Pregnenolone is the ultimate parent steroid compound. Pregnenolone is a steroid “precursor” manufactured from cholesterol, produced in the brain and the adrenal cortex. In the adrenal gland, pregnenolone is a precursor to cortisol, DHEA and progesterone. In the ovaries, pregnenolone is a precursor to estrogens and progesterone, and, in the testes, pregnenolone is a precursor to testosterone.

Progesterone is produced in the body from cholesterol and is a precursor to most of the other steroid hormones, including cortisol, estrogens and testosterone. Progesterone is made in men by the adrenal glands and testes. Progesterone is vital to good health in both women and men. It is the primary precursor of our adrenal cortical hormones and testosterone. Progesterone influences spermiogenesis, sperm capacitation/acrosome reaction and testosterone biosynthesis in the Leydig cells. Progesterone can protect against prostate cancer, inhibit 5α-redutase, block gonadotropin secretion, improve sleep pattern, help normalize blood sugar level and adipose tissue, prevent water accumulation, improve brain function, enhance the immune system, stimulate new bone formation, and maintain healthy cardiovascular, kidney, respiratory system, and thyroid hormone function.

During the aging process, progesterone level falls in men, especially after age 60. Estrogen dominance is a condition when there is insufficient progesterone in proportion to estrogen. Symptoms of estrogen dominance, which men can experience, include weight gain, bloating, mood swings, irritability, headaches, fatigue, depression, and hypoglycemia. Progesterone is the chief inhibitor of an enzyme called 5-alpha reductase that is responsible for converting testosterone to dihydrotestosterone (DHT), a linked to prostate cancer. Progesterone supplementation is important for obese men and those with family history of prostate cancer. In fatty tissue, an enzyme called aromatase converts testosterone to additional estrogen, which is believed to be involved in the abnormal growth of prostate. Progesterone is needed to counter-balance the effects of excess estrogens. When progesterone level decreases in men, the conversion from testosterone to DHT increases. When testosterone level decreases, the relative level of estradiol in men increases. Estradiol up-regulates the BCL2 gene, which can increase the risk of prostate cancer if the amount of progesterone present is inadequate to counteract the effect of BCL2 by stimulating the P53 (cancer protection) gene.

### BRIEF DESCRIPTION OF THE DRAWINGS

FIG. 1 illustrates the cholesterol-testosterone metabolic pathway.

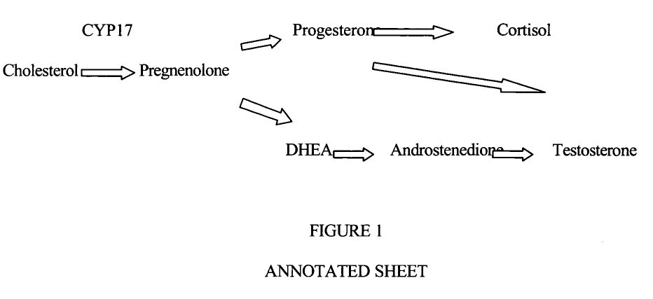

FIG. 2 illustrates the chemical intermediaries in the cholesterol-testosterone metabolic pathway.

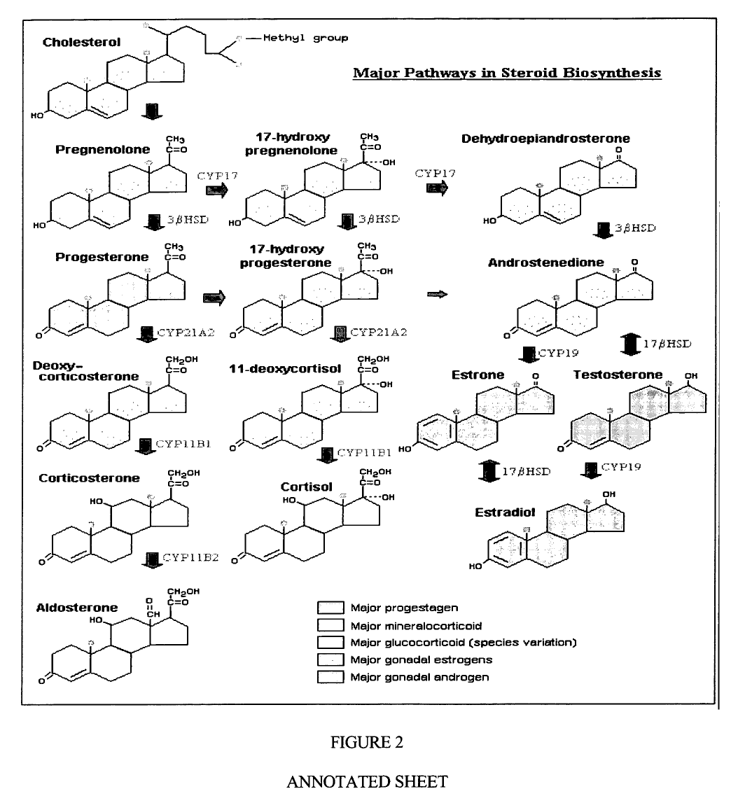

### DETAILED DESCRIPTION

I have found that one can avoid or treat Systemic Androgen Deficiency by administering an effective amount of a plant, Eurycoma longifolia. I will discuss the experimental data supporting my findings.

### EXAMPLE 1

To preliminary assess the activity of Eurycoma longifolia, an extract of it was prepared and incubated with rat testicular cell homogenate. After the incubation was completed, the samples were measured using capillary gas chromatography to measure the presence of various hormones.

Materials and Methods

Extraction of LJ100 Eurycoma Longifolia Extract:

500 g of the dried pulverized root was boiled exhaustively with 5 L of distilled water for 24 hours. The extract was concentrated to a 40% mass, allowed to cool to room temperature, filtered using a 4 micron filter, then centrifuged at 10,000 rpm for five minutes. Supernatant was collected and freeze-dried yielding yellow brown colored fine powder.

Capillary Gas Chromatography Analysis

Preparation of Buffer

Tris/sucrose buffer, PH 7.4 was used as homogenizing buffer and medium for suspending the testicular homogenate. This buffer, consisting of 0.25M sucrose, 0.05M Tris-HCL, 0.025M KCl and 0.005M MgCl₂H₂O was prepared fresh and kept cool at 4° C.

Preparation of Testicular Homogenate

Sprague Dawley albino male rats were sacrificed by stunning followed by cervical dislocation. Testes were removed quickly, pooled and homogenized in Tris/sucrose buffer (10% w/v) using homogenizer. Homogenate was then centrifuged at 1000×g (3000 rpm) for 20 minutes to remove the nuclei and cell debris. Post-nuclei suspension was used for incubation studies; a portion of the suspension was reserved for protein determination.

Note: Homogenate preparation procedures were carried out at 4° C. unless otherwise stated

Incubation Procedures

Control:

10 ml of the testes homogenate was first treated with 5 ml ethyl acetate before incubation process. Another portion of 5 ml ethyl acetate was added to terminate the reaction.

Pregnenolone:

Pregnenolone was dissolved in ethanol to make 1 mg/ml solution. 316 μl (1 μmole) was used to coat each incubation tubes. 50 μl acetone was added to each tubes followed by 10 ml of testes homogenate.

Reaction was initiated with the addition of cofactor, NADPH (12 μmole). Reaction mixture was incubated in a shaking water bath for six hours at 37° C. Reaction was terminated with the addition of 5 ml ethyl acetate.

Steroids:

10 ml testes homogenate was added to pregnenolone coated incubation tubes (prepared as described above). 1 mg of LJ100 was added to the incubation tube and mixed well.

Reaction was initiated with the addition of cofactor, ?-NAPH (12 μmole). Reaction mixture was incubated in a shaking water bath for six hours at 37° C. Reaction was terminated with the addition of 5 ml ethyl acetate.

Results

Our results are shown in the Table.

| | Eurycoma longifolia | |
|---|---|---|
| Steroid Hormone Level | Extract | Control |
| Testosterone | 3.91 ± 0.73 | 1.53 ± 0.19 |
| Progesterone | 23.62 ± 1.25 | trace |
| 17-OH Progesterone | 5.28 ± 0.46 | 0.95 ± 0.23 |
| 17-OH Pregnenolone | 0.69 ± 0.09 | 1.79 ± 0.07 |

This test shows that Eurycoma longifolia significantly increases the amount of testosterone and progesterone produced by the cell homogenate. These results also show that progesterone and 17-OH Progesterone were distinctly high in the presence of Eurycoma longifolia as compared to the control.

Both hormones were only detected in trace amount in the control. The relatively low amount of pregnenolone and 17-OH pregnenolone compared to control also suggests that Eurycoma longifolia helps in vitro to activate enzymes which convert pregnenolone into progesterone, and convert 17-OH pregnenolone into DHEA and ultimately into testosterone.

Gas capillary chromatography also was used to measure the amount of Pregnenolone metabolites in mice. These results are shown below.

ug steroid/10 mg protein

| | | | Eurycoma | |
|---|---|---|---|---|
| | | | longifolia | Eurycoma |
| Steroid Extract | Blank | Control | (2) | longifolia (3) |
| 5 alpha-androstenone | * | * | * | * |
| androstenedione | * | * | * | 0.08 |
| 17 alpha-diol, 17 beta-diol | * | * | * | * |
| Andien-beta & an-beta | 4.05 | 2.68 | 0.88 | 1.42 |
| An-alpha | 22.57 | 32.7 | 109.99 | 207.26 |
| 5-androstediol | * | * | * | * |
| 5-alpha-DHT | * | * | * | * |
| 4-androstenedione | * | * | * | 0.07 |
| Testosterone | 0.98 | 1.68 | 2.43 | 2.37 |
| 180% increase | | | | |
| 5 alpha-androstane-diol | * | * | * | * |
| 7-OH preg | 2.43 | 5.15 | 1.41 | 1.31 |
| progesterone | 3.36 | 6.39 | 12.30 | 13.20 |
| 190% increase | | | | |

These data demonstrate that there is no elevation of dihydrotestosterone with Eurycoma longifolia. The high level of Progesterone suggests Eurycoma longifolia blocks the enzyme 5 alpha-reductase and inhibits the conversion of testosterone to dihydrotestosterone (DHT), which is only seen in trace amounts in this study.

The relatively high level of Progesterone and Testosterone shown in this study also confirm that a healthy testosterone/estradiol level in men might be able to be maintained by using Eurycoma longifolia, to reduce the risk of prostate cancer. 17α-diol and 17 β-diol is only seen in trace amounts in this study.

| | Control | Prenenolone | Eurycoma longifolia |
|---|---|---|---|
| An α | 2.52 | 1.62 | |
| * | +0.12 | +0.28 | +0.44 |
| An β | 2.64 | 1.99 | 0.38 |
| Andien β | +0.09 | +0.44 | +0.11 |

Besides the steroid hormones, gas capillaries analysis also detected the presence of other steroid metabolites that were not involved in steroid hormone biosynthesis. These metabolites include an α, an β, and andien β; belong to the 16-androstenes steroid family. 16-androstenes steroids, also known as pheromones are axillary secretion responsible in the synthesis of odor. Apart of being the major precursor for androgen biosynthesis, pregnerblone also acted as the precursor for the synthesis of pheromones. Pregnenolone will be converted to andien β. An α plays an important role in communication, psychological and sexual behavior both in human and animals. Gas capillaries results showed that the production of an α is higher compared to an β. This study shows that Eurycoma longifolia is not only capable of increasing testosterone production, but it also influences the synthesis of pheromones.

### EXAMPLE 2

Eurycoma longifolia treatment on testosterone concentration in rat Leydig Cells, as measured by in Enzyme Linked Immuno-Sorbent Assay (ELISA) Analysis. Rat Leydig cells were incubated with either Hcg, Lac or Eurycoma longifolia. After incubation, the concentration of testosterone was measured. Five×10 4 cells/100 ul of rat leydig cells were used. 75 ug/ml of Eurycoma longifolia were added into cell cultures medium. Cells were treated with the samples and controls and incubated for an hour with 5% CO2 at 37 C. The cells were then assayed by a standard immunology assay procedure, for the determination of testosterone concentration.

| | Testosterone | |
|---|---|---|
| Treatment | Concentration (pg/ml) | % increase |
| Control | 8.92 ± 1.68 | |
| Hcg (−0.05 IU/ml) | 13.14 ± 2.61 | 47.27 |
| Lac (10 ul/ml) | 13.03 ± 3.10 | 46.11 |
| Eurycoma longifolia | 19.38 ± 2.70 | 119.77 |

These data show a significant increase in testosterone concentration following incubation with Eurycoma longifolia compared to the control and to the two positive controls. The result suggest that Eurycoma longifolia has the capability to elevate testosterone concentration in the Leydig cells up to 120%.

### EXAMPLE 3

### Gene Expression Analysis

Gene expression analysis using Real Time Reverse Transcription Polymerase Chain Reaction (RT-PCR) is based on detection and quantitation of a fluorescent signal, which increases in direct proportion to the amount of PCR product n a reaction. In this study, the signal used is a double-stranded DNA specific fluoresce dye (SYBR Green 1). Real Time RT-PCR allows researcher to examine the relative level of gene expression and variation between samples. The objective of this analysis is to observe the effect of Eurycoma longifolia towards gene expression level. As for this study, the genes of interest are the genes involved in testosterone/androgen biosynthesis pathways and also spermatogenesis.

Prior to Real Time RT-PCR, rat Leydig cells were given the same treatment and incubation as in the EIA experiment. At the end of the incubation, culture media were removed and RNA was extracted from the cells. Total RNA extracted was purified, Dnased and later used as a template for cDNA synthesis through RT-PCR method. The cDNA produced from this step will be used as a template in Real Time RT-PCR reaction. One important criterion in Real Time analysis is the primer selection and in this experiment, primers were selected based on the gene of the enzymes involved in testosterone biosynthesis pathways. From the pathways, 2 most important enzymes were chosen, i.e., the CYP17 (17, 20 lyase/17α-hydroxylase) and the CYP19 (aromatase). Apart from the two target genes, 5sRNA gene was chosen as a housekeeping gene.

The first step in calculation the amount of genes being expressed is by obtaining the data graph from each RT-PCR reaction (including the standards for target genes and housekeeping genes, treated and untreated samples). Threshold cycle (C?) values from the data graph will show the exact initial time where the RT-PCR reaction product is being released. In order to determine the amount of product released from the reaction, the C? values against a dilution series with arbitrary unit or against the logarithm of the initial known copy number.

The housekeeping gene acts as an endogenous control in the assay to correct any sample to sample variation or errors in sample quantitation. Raw values obtained from the standard curve for each treated or nontreated samples will be subjected to normalization by dividing them with the raw values for the housekeeping gene from corresponding RT-PCR reaction. Comparison of the normalized values for treated sample with normalized values for non treated (control) sample will give us the relative value i.e. the ration of the target gene expression level. Values greater than 1 indicate positive effect of the treatment/sample in influencing the gene expression level (up-regulation) whereas values less or equal to 1 reflect suppression in the gene expression level (down-regulation).

Relative Values for CYP17 Gene following Incubation with Eurycoma longifolia

| | Relative Value |
|---|---|
| hCG | 1.57 ± 0.282 |
| Eurycoma longifolia | 3.807 ± 0.590 |

Relative values were obtained by dividing the normalized value of each treated samples with the normalized control value for each corresponding group. Relative value is the means of triplicate incubations with triplicate RT-PCR reactions in each incubation ±SD. All values were statistically evaluated using SPSS version 11.0; differences were significant at p<0.01

SD—standard Deviation

CYP 17 (17a-hydroxylase/17, 20 lyase) involves in the early stage of steroid biosynthesis. Results from this study showed that Eurycoma longifolia significantly increased the expression of CYP17 gene compared to the positive control (hCG). The observed effect towards CYP17 gene expression might suggests that more of this enzyme is being produced, which will enhance the metabolism of pregnenolone and 17-OH pregnenolone to yield more dehyroepiandrosterone (DHEA) as well as the metabolism of progesterone and 17-OH progesterone to 4-androstenedione. This process is important to testosterone. This result also proved the ability of Eurycoma longifolia to elevate testosterone production.

### EXAMPLE 4

### Effects Toward Gene Expression Level—CYP 17

Steroid synthesis and sperm production represent the main characteristics of the mammalian testis. These functions are controlled by gonadotropins and several hormones, obviously testosterone (androgen) and oestrogen. This study has reported the influence of Eurycoma longifolia toward testosterone production and several sperm characteristics. In order to exert these biological effects, the hormones in the testis should interact with their specific receptors which in turn will induce the transcription of specific genes. The aim of this present study is to confirm the effect of Eurycoma longifolia as seen earlier and to investigate its effect at gene level. The genes selected for this particular study were selected based on the enzymes involved in steroid biosynthesis pathways; mainly the enzyme involved in ?4 and ?5 pathways. These include CYP17 and CYP 19, the enzyme that involved in spermatogenesis i.e. enzyme that converts testosterone into oestrogen.

CYP17

CYP17 or 17a-hydroxylase/17,20 lyase involves in the early stage of steroid biosynthesis in both ?4 and ?5. These enzymes convert pregnenlonoe into 17a-hydroxypregnenolone and to dehydroepiandrosterone (DHEA) (?5 pathway) or from progesterone to 17 a-hydroxyprogesterone and 4-androstenedione (?4 pathway). The end product i.e. DHEA and 4 androstenedione will be directly converted to testosterone, the major androgen in male individuals.

RT-PCR analysis from this study observed an obvious increase in the CYP17 gene expression level upon incubation with Eurycoma longifolia. Relative values were eight times higher than the control with the presence of Eurycoma longifolia. These results could suggest that Eurycoma longifolia have the potential to influence up-regulation of the CYP17 gene expression and therefore leads to the elevation of testosterone production. This also suggests that Eurycoma longifolia could be useful in hormone therapy treatment in order to correct erratic testosterone level or testosterone deficiency problems.

### EXAMPLE 5

### Eurycoma longifolia Saliva Testosterone Test

The amount of testosterone was measured using a saliva testosterone test. This test was administered to 9 Individuals, ranging from 26 to 52 years of age. Volunteers 1-5 are athletes; Volunteers 6-9 do not exercise on a regular basis.

Each test subject was administered Eurycoma longifolia, two 50 mg capsules (a total of 100 mg), twice a day (morning & evening) (for a total of 200 mg per day) for 10 days.

The results are shown in the Table. For comparison, the normal range for an athlete is 800=150 ng of testosterone per dl of blood.

| | | pre treatment | after treatment | % |
|---|---|---|---|---|
| Volunteer | age | ng/dl blood | ng/dl blood | Increase |
| 1 | 26 | 860 = 30 | 1,650 = 50 | 91.86% |
| 2 | 28 | 580 = 30 | 985 = 35 | 69.83% |
| 3 | 35 | 875 = 40 | 1,576 = 60 | 80.11% |
| 4 | 24 | 950 = 45 | 2,210 = 55 | 132.63% |
| 5 | 29 | 755 = 30 | 1,345 = 35 | 78.15% |
| 6 | 48 | 650 = 20 | 875 = 30 | 34.62% |
| 7 | 52 | 450 = 25 | 765 = 35 | 70.00% |
| 8 | 50 | 585 = 25 | 875 = 35 | 49.57% |
| 9 | 42 | 350 = 30 | 480 = 35 | 37.14% |

This data provides preliminary results, leaving open the possibility for more productive work to be carried out. This data, however, shows significant increases in testosterone levels, as measured by a saliva test, with the administration of Eurycoma longifolia. For all subjects, Eurycoma longifolia increases the level of testosterone an average of 71.56%. Among athletic subjects, Eurycoma longifolia increases the level of testosterone even more, an average of 90.52%.

Volunteers 1-5 are athletes—data are an average of measurements at three different times

Volunteers 6-9 do not exercise on a regular basis

Other Applications

1. Activates enzymatic sequence of Androgen Steroid Biosynthesis:
(a) Break down Cholesterol to Pregnelolone, which is further converted to Progesterone and Testosterone (Reduce Cholesterol and Pregnenolone)
(b) Activates CYP17 enzymes, which enhanced the metabolism of pregnenolone and 17-OH pregnenolone to yield more dehyroepiandrosterone (DHEA).
(c) Increase dehydroepiandrosterone (DHEA)
(d) Increase Progesterone

2. Decrease Sex Hormone Binding Globulin

3. Increase Free Testosterone (FT)

4. Increase DHEA

5. Increase Progesterone level:
blocks enzyme 5 alpha-reductase, inhibits the conversion of testosterone to dihydrotestosterone (DHT),
maintain healthy level of testosterone/estradiol level and prostate health.
Helps normalize blood sugar levels
Helps use fat for energy
Prevents water accumulation (acts as mild diuretic)
Helps (normalizes) thyroid hormone function
Stimulates new bone formation (osteoporosis protection and even reversal)
Improves brain function, has antidepressant properties
Improves skin problems including acne, seborrhea, rosacea, psoriasis
Improves sleep pattern
Improves libido.

6. Increase the synthesis of Pheromone An-alpha responsible for Sexual communication and behavior

7. Increase ATP (adenosine triphosphate) basic energy unit production

8. Increase cGMP and cAMP leads to vasodilation in penile tissue

9. Increase Insulin-like Growth Factor-1 (IGF1) level/Anti-aging/human
Regulates cellular growth and development
IGF-1 stimulates muscle bulk and lean body mass
helps burn fat
promotes healthy blood sugar level
decreases LDL Cholesterol.

10. Weight Management: Increase Tyroxin level leads to increase metabolism rate, increase calories burned, improve physical development, and decrease fatigue.

11. Modulate Cortisol: Boost Immune Function during illnesses and body defense during health crisis.

12. Improved Sports Performance:
(a) Increase FT leads to improved energy as adenosine triphosphate (ATP) production increases
(b) Increase FT leads to increment in fat free mass, greater decrement in body fat, increase in muscle mass and strength
(c) Improved recovery time
(d) Increase Tyroxin level leads to increase Basal Body Metabolism Rate, increase calories burned, improve physical development, decrease fatigue and sleeps disorder, and improving overall quality of life.
(e) Increase IFG-1 level, a natural anabolic growth factor that stimulates muscle bulk and lean body mass, and helps burn fat.

13. For treatment of Andropause/symptomatic late-onset hypogonadism/somatopause: Decrease Sex Hormone Binding Globulin that result in increase Free Testosterone (FT), leads to
(a) Enhancement Sexual Performance:
   (i) Increase the synthesis of Pheromone An-? Responsible for Sexual communication and behavior
   (ii) Increase cGMP and cAMP that causes relaxation of erectile tissue, increase vasodilation that leads to penile erection
(b) Stimulate production of red blood cell in bone marrow bone formation
(c) Regulates Cholesterol: Increase lipid metabolism leads to improvement in high-density lipoprotein(HDL Cholesterol-good cholesterol). Lower HDL are normally associated with a lower risk of heart attack.
(d) Increased carbohydrate metabolism
(e) Promote hair growth
(f) Promote immune response
(g) Promote spermatogenesis
(h) Inhibits osteoclasts that enhance bone breakdown

14. For treating type-2 diabetes: lowering the blood glucose levels

15. For treating Metabolic Syndrome X

16. For reducing estrogen-dominance in men.

Summary

My invention may be used to make a Drug (as that term is defined in the Federal Food, Drug & Cosmetic Act). Alternatively, my invention may be used to make a Food (as that term is defined in the Federal Food, Drug & Cosmetic Act) or a Dietary Supplement (as that term is defined in the Federal Food, Drug & Cosmetic Act), or a Cosmetic. Thus, my claims can cover a pill used as a Drug, or a pill used as a Dietary Supplement, or a Food such as a meal-replacement bar a or a nutrition shake mix, or a Cosmetic skin cream.

In the claims appended, I use the term “treating” to encompass both administering a medical therapy (as in administering a pharmaceutical) and preserving a healthy normal physiological state (as in taking a dietary supplement). Depending on the esired use, one can use anywhere from about 0.25 grams to about 3.0 grams per day as a useful dosage.

I use the term “Systemic Androgen Deficiency” to mean a systemic level of androgen which is lower than is considered desirable. This can be due to androgen deficiency due to andropause, hypogonadalism, or a relative excess of Sex Hormone Binding Globulin. Alternatively, it may be due to an impairment in the conversion of cholesterol into pregnenolone, or pregnenolone into progesterone, dehydroepiandrosterone, cortisol or testosterone. It may adversely impact CYP17 or leutenizing hormone activity. It may evidence itself clinically as an elevated cholesterol level, or a decrease in the ratio of high density lipoprotein to low density lipoprotein, or a decrease in the level of insulin growth factor-1.

Given the discussion above, one of skill in the art can readily modify my invention. For example, one may administer Eurycoma longifolia as a tablet, or gelatin capsule, or even as a liquid. Similarly, while I discuss twice-daily dosing, one could develop a once-daily or “extended-release” dosing. Thus, while I discuss my Examples above in some detail, I intend the legal coverage of my patent to be defined not by the specific examples above, nor the Abstract, but by the literal coverage of the appended Claims, and allowable legal equivalents thereof.

1. A method for treating Systemic Androgen Deficiency, said method comprising:
a. diagnosing in a patient Systemic Androgen Deficiency; and
b. administering to said patient Eurycoma longifolia in an amount effective to ameliorate said Systemic Androgen Deficiency.

2. The method of claim 1, said amount effective comprising from about 0.5 mg to about 10.0 mg of per kilogram of patient body weight per day.

3. The method of claim 2, wherein said Systemic Androgen Deficiency comprises andropause.

4. The method of claim 2, wherein said Systemic Androgen Deficiency comprises androgen deficiency.

5. The method of claim 2, wherein said Systemic Androgen Deficiency comprises hypogonadism.

6. The method of claim 2, wherein said amount is effective to treat or prevent erectile dysfunction.

7. The method of claim 1, wherein said amount comprises an amount effective to decrease available Sex Hormone Binding Globulin in an amount sufficient to increase available testosterone.

8. The method of claim 1, wherein said amount comprises an amount effective to modulate the activity of CYP17.

9. The method of claim 1, wherein said amount comprises an amount effective to modulate the activity of leutenizing hormone.

10. The method of claim 1, wherein said amount comprises an amount effective to modulate the activity of CYP17.

11. The method of claim 1, wherein said amount comprises an amount effective to modulate the conversion of cholesterol into pregnenolone.

12. The method of claim 1, wherein said amount comprises an amount effective to modulate the conversion of pregnenolone into progesterone.

13. The method of claim 1, wherein said amount comprises an amount effective to modulate the conversion of pregnenolone into dehydroepiandrosterone.

14. The method of claim 1, wherein said amount comprises an amount effective to modulate the conversion of pregnenolone into cortisol.

15. The method of claim 1, wherein said amount comprises an amount effective to modulate cortisol.

16. The method of claim 1, wherein said amount comprises an amount effective to modulate the conversion of pregnenolone into testosterone.

17. The method of claim 1, wherein said amount comprises an amount effective to increase testosterone.

18. The method of claim 1, wherein said amount comprises an amount effective to increase progesterone.

19. The method of claim 1, wherein said amount comprises an amount effective to increase DHEA.

20. The method of claim 1, wherein said amount comprises an amount effective to increase said patient's ratio of high density lipoprotein to low density lipoprotein.

21. The method of claim 1, wherein said amount comprises an amount effective to increase said patient's level of insulin growth factor-1.

22. The method of claim 1, wherein said amount comprises an amount effective to increase said patient's level of pheromone An-alpha.

23. The method of claim 1, wherein said amount comprises an amount effective to inhibit 5α-reductase in vivo.

24. A method to increase a patient's basal metabolic rate, said method comprising administering to said patient Eurycoma longifolia in an amount effective to increase said patient's basal metabolic rate.
